(19) United States
(12) Patent Application Publication
(10) Pub. No.: US 2010/0204167 A1
(43) Pub. Date: Aug. 12, 2010

(54) ACTIVE COMPOUND COMBINATIONS HAVING INSECTICIDAL AND ACARICIDAL PROPERTIES

(76) Inventors: Reiner Fischer, Monheim (DE); Wolfram Andersch, Bergisch Gladbach (DE); Thomas König, Hilden (DE); Anton Kraus, Leichlingen (DE); Emmanuel Salmon, Koln (DE); Heike Hungenberg, Langenfeld (DE)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/373,188

(22) PCT Filed: Jul. 6, 2007
(86) PCT No.: PCT/EP07/05994

§ 371 Date: Oct. 16, 2009

(30) Foreign Application Priority Data
Jul. 11, 2006 (DE) 10 2006 031 975.3

(51) Int. Cl. A61K 31/7048; A01N 43/04; A01N 43/713; A01N 43/58; A01N 43/54; A01N 43/56; A01N 43/38; A01P 7/02; A01P 7/04

(52) U.S. Cl. 514/28; 514/30; 514/183; 514/247; 514/266.3; 514/267; 514/406; 514/407; 514/409

(57) ABSTRACT

The novel active compound combinations comprising a compound of the formula (I-1) or (I-2) and the active compounds (1) to (26) listed in the description have very good insecticidal and acaricidal properties.

The present invention relates to novel active compound combinations consisting, firstly, of known cyclic ketoenols and, secondly, of other known insecticidally active compounds, which active compound combinations are highly suitable for controlling animal pests such as insects and unwanted acarids.

It is already known that certain cyclic ketoenols have herbicidal, insecticidal and acaricidal properties. The activity of these compounds is good; however, it is sometimes unsatisfactory at low application rates.

Known to have insecticidal and/or acaricidal activity are 1H-3-arylpyrrolidine-2,4-dione derivatives from WO 98/05638 and their cis isomers from WO 04/007448.

Also known are mixtures of compounds from WO 98/05638 with other insecticides and/or acaricides: WO 01/89300, WO 02/00025, WO 02/05648, WO 02/17715, WO 02/19824, WO 02/30199, WO 02/37963, WO 05/004603, WO 05/053405, WO 06/089665, DE-A-10342673. However, the activity of these mixtures is not always satisfactory.

It has now been found that active compound combinations comprising compounds of the formula (I-1) or (I-2)

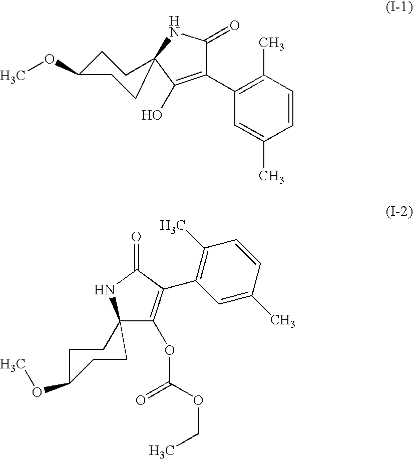

and acaricidally active compounds, preferably
(1) the phenylhydrazine derivative of the formula

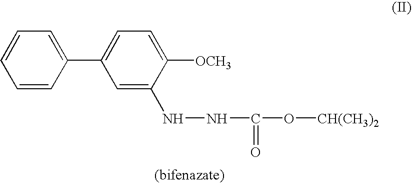

known from WO 93/10 083

and/or
(2) the macrolide with the common name
abamectin (III)

known from DE-A-27 17 040

and/or
(3) the naphthalenedione derivative of the formula

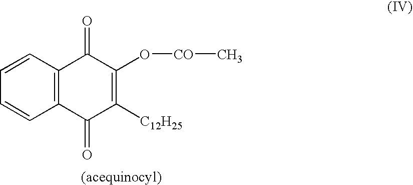

known from DE-A-26 41 343

and/or
(4) the pyrrole derivative of the formula

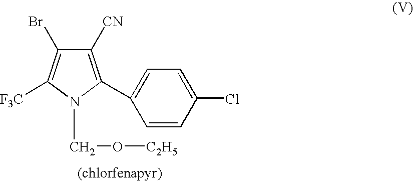

known from EP-A-347 488

and/or
(5) the thiourea derivative of the formula

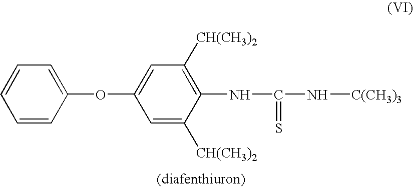

known from EP-A-210 487

and/or
(6) the oxazoline derivative of the formula

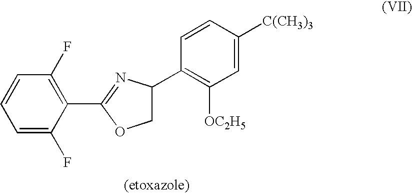

known from WO 93/22 297

and/or
(7) an organotin derivative of the formula

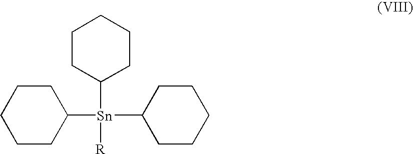

in which

R represents

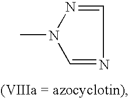

known from The Pesticide Manual, 9th edition, p. 48

or

R represents —OH
(VIIIb=cyhexatin),
known from U.S. Pat. No. 3,264,177
   and/or
   (8) the pyrazole derivative of the formula

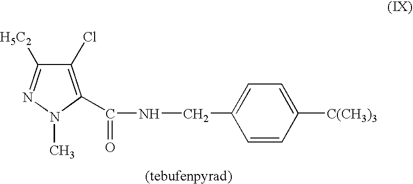

known from EP-A-289 879

and/or
(9) the pyrazole derivative of the formula

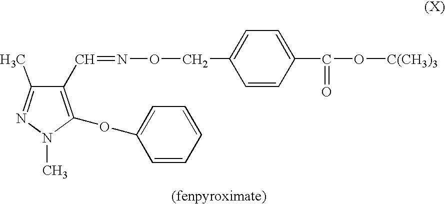

known from EP-A-234 045

and/or
(10) the pyridazinone derivative of the formula

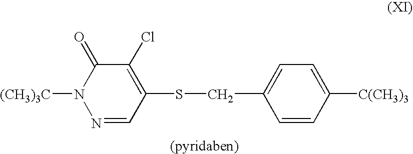

known from EP-A-134 439

and/or
(11) the tetrazine derivative of the formula

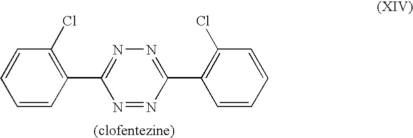

known from EP-A-005 912

and/or
(12) the organotin derivative of the formula

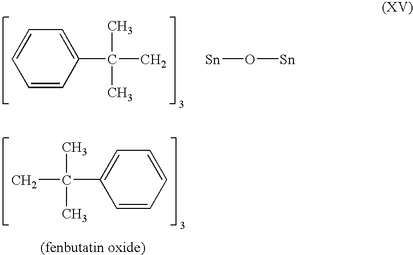

known from DE-A-2 115 666

and/or
(13) the sulfenamide of the formula

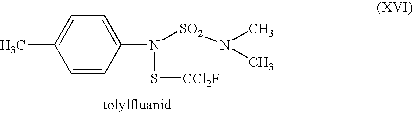

known from The Pesticide Manual, 11th edition, 1997, page 1208

(14) and/or the pyrimidylphenol ethers

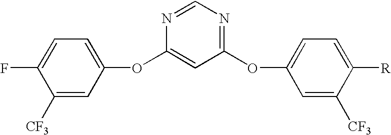

R═Cl (XVII); (4-[(4-chloro-α,α,α-trifluoro-3-toly)oxy]-6-[(α,α,α-4-tetrafluoro-3-tolyl)oxy]pyrimidine)
R═NO₂ (XVIII); 4-[(4-chloro-α,α,α-trifluoro-3-tolyl)oxy]-6-[(α,α,α-trifluoro-4-nitro-3-tolyl)oxy]pyrimidine
R═Br (XIX); 4-[(4-chloro-α,α,α-trifluoro-3-tolyl)oxy]-6-[(α,α,α-tri-fluoro-4-bromo-3-tolyl)oxy]pyrimidine
known from WO 94/02 470, EP-A-883 991
and/or
   (15) the macrolide of the formula

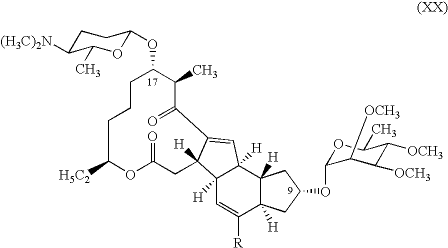

(spinosad) a mixture of preferably

85% spinosyn A R═H

15% spinosyn B R═CH₃

known from EP-A-375 316

and/or

(16) ivermectin (XXI)

known from EP-A-001 689

and/or

(17) milbemectin (XXII)

known from The Pesticide Manual, 11th edition, 1997, p. 846

and/or

(18)

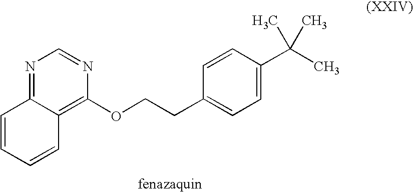

known from EP-A-326 329

and/or

(19)

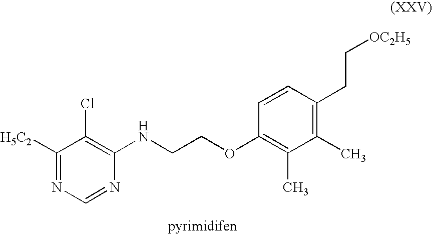

known from EP-A-196 524

and/or

(20)

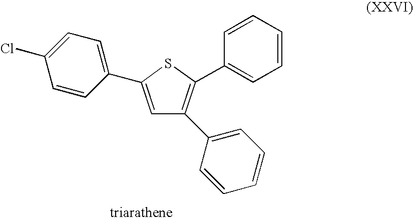

known from DE-A-2 724 494

and/or

(21)

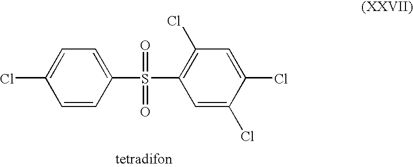

known from U.S. Pat. No. 2,812,281

and/or

(22)

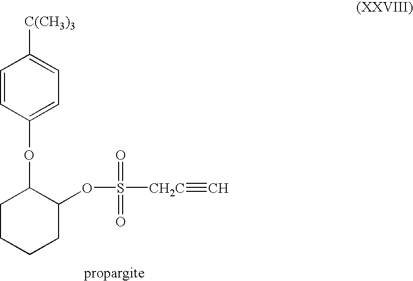

known from U.S. Pat. No. 3,272,854

and/or

(23)

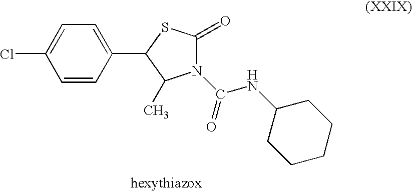

known from DE-A-3 037 105

and/or

(24)

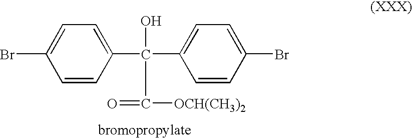

known from U.S. Pat. No. 3,784,696

and/or

(25)

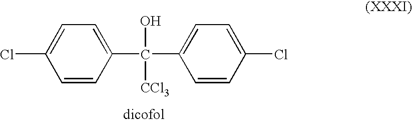

known from U.S. Pat. No. 2,812,280

and/or

(26)

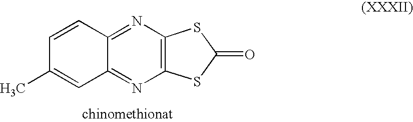

known from DE-A-1 100 372

have very good insecticidal and/or acaricidal properties.

Surprisingly, the insecticidal and/or acaricidal activity of the active compound combinations according to the invention is considerably better than the activities of the prior-art active compound combination from WO 02/037963 consisting of cis/trans isomer mixtures of the formula I-1-a or I-2-a and an active compound of the compounds 1 to 26.

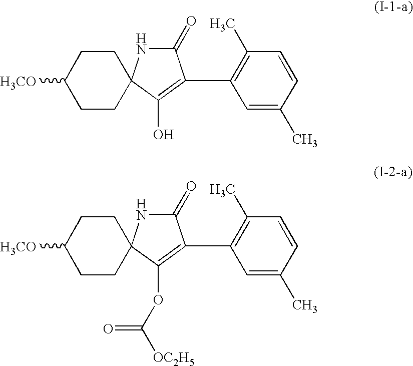

Preference is given to active compound combinations comprising the compound of the formula (I-1) and at least one of the active compounds of the compounds 1 to 26.

Preference is given to active compound combinations comprising the compound of the formula (I-2) and at least one of the active compounds of the compounds 1 to 26.

In addition, the active compound combinations may also comprise further fungicidally, acaricidally or insecticidally active additives.

The improved activity becomes particularly evident when the active compounds in the active compound combinations according to the invention are present in certain weight ratios. However, the weight ratios of the active compounds in the active compound combinations can be varied within a relatively wide range. In general, the combinations according to the invention comprise active compounds of the formula (I-1) or (I-2) and the mixing partner in the preferred and particularly preferred mixing ratios stated in the table below:
the mixing ratios are based on weight ratios. The ratio is to be understood as active compound of the formula (I-1):mixing partner or formula (I-2):mixing partner

| | Preferred | Particularly preferred |
|---|---|---|
| Mixing partner | mixing ratio | mixing ratio |
| bifenazate | 5:1 to 1:25 | 5:1 to 1:5 |
| abamectin | 125:1 to 1:5 | 25:1 to 1:1 |
| acequinocyl | 25:1 to 1:25 | 5:1 to 1:5 |
| chlorfenapyr | 25:1 to 1:25 | 5:1 to 1:5 |
| diafenthiuron | 25:1 to 1:25 | 5:1 to 1:5 |
| etoxazole | 25:1 to 1:5 | 5:1 to 1:5 |
| azocyclotin | 25:1 to 1:25 | 5:1 to 1:5 |
| cyhexatin | 25:1 to 1:25 | 5:1 to 1:5 |
| tebufenpyrad | 25:1 to 1:25 | 5:1 to 1:5 |
| fenpyroximate | 25:1 to 1:25 | 5:1 to 1:5 |
| pyridaben | 25:1 to 1:25 | 5:1 to 1:5 |
| clofentezine | 25:1 to 1:25 | 5:1 to 1:5 |
| fenbutatin oxide | 10:1 to 1:10 | 5:1 to 1:5 |
| tolylfluanid | 5:1 to 1:50 | 1:1 to 1:5 |
| pyrimidylphenol ethers | 10:1 to 1:10 | 5:1 to 1:5 |
| (XVII-XIX) | | |
| spinosad | 25:1 to 1:25 | 5:1 to 1:5 |
| ivermectin | 125:1 to 1:5 | 10:1 to 1:1 |
| milbemectin | 125:1 to 1:5 | 10:1 to 1:1 |
| fenazaquin | 25:1 to 1:25 | 5:1 to 1:5 |
| pyrimidifen | 25:1 to 1:5 | 5:1 to 1:1 |
| triarathene | 5:1 to 1:20 | 1:1 to 1:10 |
| tetradifon | 10:1 to 1:10 | 5:1 to 1:5 |
| propargite | 10:1 to 1:25 | 5:1 to 1:5 |
| hexythiazox | 10:1 to 1:5 | 5:1 to 1:2 |
| bromopropylate | 10:1 to 1:10 | 5:1 to 1:5 |
| dicofol | 10:1 to 1:10 | 5:1 to 1:5 |
| chinomethionat | 10:1 to 1:10 | 5:1 to 1:5 |

The active compound combinations according to the invention are suitable for controlling animal pests, preferably arthropods and nematodes, in particular insects and/or arachnids, found in viticulture and the cultivation of fruit, in horticulture, in agriculture, in animal health, in forests, in the protection of stored products and in the protection of materials and also in the hygiene sector. They are active against normally sensitive and resistant species, and against all or individual developmental stages. The abovementioned pests include:

From the order of the Isopoda, for example, Oniscus asellus, Armadillidium vulgare, Porcellio scacalc.

From the order of the Diplopoda, for example, Blaniulus guttulatus.

From the order of the Chilopoda, for example, Geophilus carpophagus, Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Acheta domesticus, Gryllotalpa spp., Locusta migratoria migratorioides, Melanoplus spp., Schistocerca gregaria.

From the order of the Blattaria, for example, Blatta orientalis, Periplaneta americana, Leucophaea maderae, Blattella germanica.

From the order of the Dermaptera, for example, Forficula auricularia.

From the order of the Isoptera, for example, Reticulitermes spp.

From the order of the Phthiraptera, for example, Pediculus humanus corporis, Haematopinus spp., Linognathus spp., Trichodectes spp., Damalinia spp.

From the order of the Thysanoptera, for example, Hercinothrips femoralis, Thrips tabaci, Thrips palmi, Frankliniella accidentalis.

From the order of the Heteroptera, for example, Eurygaster spp., Dysdercus intermedius, Piesma quadrata, Cimex lectularius, Rhodnius prolixus, Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp., Psylla spp.

From the order of the Lepidoptera, for example, Pectinophora gossypiella, Bupalus piniarius, Chematobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp., Oulema oryzae.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica, Lissorhoptrus oryzophilus.

From the order of the Hymenoptera, for example, Diprion spp., Hoplocampa spp., Lasius spp., Monomorium pharaonis, Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp., Liriomyza spp.

From the order of the Siphonaptera, for example, Xenopsylla cheopis, Ceratophyllus spp.

From the class of the Arachnida, for example, Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp., Brevipalpus spp.

The plant-parasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp., Bursaphelenchus spp.

The active compound combinations can be converted into the customary formulations such as solutions, emulsions, wettable powders, suspensions, powders, dusts, pastes, soluble powders, granules, suspension-emulsion concentrates, natural and synthetic materials impregnated with active compound, and microencapsulations in polymeric materials.

These formulations are produced in a known manner, for example by mixing the active compounds with extenders, that is, liquid solvents and/or solid carriers, optionally with the use of surfactants, that is, emulsifiers and/or dispersants, and/or foam formers.

If the extender used is water, it is also possible, for example, to use organic solvents as cosolvents. The following are essentially suitable as liquid solvents: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, or else water.

Suitable solid carriers are:

for example ammonium salts and ground natural minerals such as kaolins, clays, talc, chalk, quartz, attapulgite, montmorillonite or diatomaceous earth, and ground synthetic minerals such as highly disperse silica, alumina and silicates; suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, corn cobs and tobacco stalks; suitable emulsifiers and/or foam formers are: for example nonionic and anionic emulsifiers such as polyoxyethylene fatty acid esters, polyoxyethylene fatty alcohol ethers, for example alkylaryl polyglycol ethers, alkylsulfonates, alkyl sulfates, arylsulfonates, or else protein hydrolyzates; suitable dispersants are: for example lignosulfite waste liquors and methylcellulose.

Tackifiers such as carboxymethylcellulose and natural and synthetic polymers in the form of powders, granules or lattices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic colorants such as alizarin colorants, azo colorants and metal phthalocyanine colorants, and trace nutrients such as salts of iron, manganese, boron, copper, cobalt, molybdenum and zinc.

The formulations generally comprise between 0.1 and 95% by weight of active compound, preferably between 0.5 and 90%.

The active compound combinations according to the invention can be present in commercially available formulations and in the use forms, prepared from these formulations, as a mixture with other active compounds, such as insecticides, attractants, sterilants, bactericides, acaricides, nematicides, fungicides, growth-regulating substances or herbicides. The insecticides include, for example, phosphates, carbamates, carboxylates, chlorinated hydrocarbons, phenylureas and substances produced by microorganisms, inter alia.

Mixtures with other known active compounds such as herbicides or with fertilizers and growth regulators are also possible.

When used as insecticides, the active compound combinations according to the invention can furthermore be present in their commercially available formulations and in the use forms, prepared from these formulations, as a mixture with synergists. Synergists are compounds which increase the action of the active compounds, without it being necessary for the synergist added to be active itself.

The active compound content of the use forms prepared from the commercially available formulations can vary within wide limits. The active compound concentration of the use forms can be from 0.0000001 to 95% by weight of active compound, preferably between 0.0001 and 1% by weight.

The compounds are employed in a customary manner appropriate for the use forms.

According to the invention, it is possible to treat all plants and parts of plants. Plants are to be understood here as meaning all plants and plant populations such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including the plant cultivars which can or cannot be protected by plant breeders' certificates. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested plants and vegetative and generative propagation material, for example seedlings, tubers, rhizomes, cuttings and seeds.

The treatment according to the invention of the plants and parts of plants with the active compounds is carried out directly or by action on their environment, habitat or storage area according to customary treatment methods, for example by dipping, spraying, evaporating, atomizing, broadcasting, brushing-on and, in the case of propagation material, in particular in the case of seeds, furthermore by one- or multi-layer coating.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (Genetic Modified Organisms), and parts thereof are treated. The terms “parts”, “parts of plants” and “plant parts” have been explained above.

Particularly preferably, plants of the plant cultivars which are in each case commercially available or in use are treated according to the invention.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferred and to be treated according to the invention include all plants which, in the genetic modification, received genetic material which imparts particularly advantageous useful traits to these plants. Examples of such traits are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defense of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), corn, soybeans, potatoes, cotton, oilseed rape and also fruit plants (with the fruits apples, pears, citrus fruits and grapes), and particular emphasis is given to corn, soybeans, potatoes, cotton and oilseed rape. Traits that are particularly emphasized are the increased defense of the plants against insects by toxins formed in the plants, in particular those formed by the genetic material from Bacillus Thuringiensis (for example by the genes CryIA(a), CryIA(b), CryIA(c), CryIIA, CryIIIA, CryIIIB2, Cry9c Cry2Ab, Cry3Bb and CryIF and also combinations thereof) (hereinbelow referred to as “Bt plants”). Traits that are furthermore particularly emphasized are the increased tolerance of the plants to certain herbicidally active compounds, for example imidazolinones, sulfonylureas, glyphosate or phosphinotricin (for example the “PAT” gene). The genes in question which impart the desired traits can also be present in combination with one another in the transgenic plants. Examples of “Bt plants” which may be mentioned are corn varieties, cotton varieties, soybean varieties and potato varieties which are sold under the trade names YIELD GARD® (for example corn, cotton, soybeans), KnockOut® (for example corn), StarLink® (for example corn), Bollgard® (cotton), Nucotn® (cotton) and NewLeaf® (potato). Examples of herbicide-tolerant plants which may be mentioned are corn varieties, cotton varieties and soybean varieties which are sold under the trade names Roundup Ready® (tolerance to glyphosate, for example corn, cotton, soybean), Liberty Link® (tolerance to phosphinotricin, for example oilseed rape), IMI® (tolerance to imidazolinones) and STS® (tolerance to sulfonylureas, for example corn). Herbicide-resistant plants (plants bred in a conventional manner for herbicide tolerance) which may be mentioned include the varieties sold under the name Clearfield® (for example corn). Of course, these statements also apply to plant cultivars having these or still-to-be-developed genetic traits, which plant cultivars will be developed and/or marketed in the future.

The plants listed can be treated according to the invention in a particularly advantageous manner with the active compound mixture according to the invention. The preferred ranges stated above for the mixtures also apply to the treatment of these plants. Particular emphasis is given to the treatment of plants with the mixtures specifically mentioned in the present text.

The expected action for a given combination of two active compounds can be calculated as follows, according to S. R. Colby, Weeds 15 (1967), 20-22:

### If

X is the kill rate, expressed as a percentage of the untreated control, when employing active compound A at an application rate of m g/ha or in a concentration of m ppm,
Y is the kill rate, expressed as a percentage of the untreated control, when employing active compound B at an application rate of n g/ha or in a concentration of n ppm and
E is the kill rate, expressed as a percentage of the untreated control, when employing active compounds A and B at application rates of m and n g/ha or in a concentration of m and n ppm,
   then

$$E = X + Y - \frac{X \cdot Y}{100}$$

If the actual kill rate exceeds the calculated value, the kill of the combination is superadditive, i.e. a synergistic effect is present. In this case, the actually observed kill rate must exceed the value calculated using the above formula for the expected kill rate (E).

### EXAMPLE A

Aphis gossypii Test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amount of solvent and the stated amount of emulsifier, and the concentrate is diluted with emulsifier-containing water to the desired concentration.

Cotton leaves (Gossypium herbaceum) which are heavily infested by the cotton aphid (Aphis gossypii) are treated by being dipped into the active compound preparation of the desired concentration.

After the desired period of time, the kill in % is determined. 100% means that all aphids have been killed; 0% means that none of the aphids have been killed. The kill rates determined are entered into Colby's formula.

In this test, for example, the following active compound combinations in accordance with the present application show a synergistically enhanced activity compared to the active compounds applied individually:

TABLE A1
Plant-damaging insects
Aphis gossypii test

| | Concentration | Kill | |
|---|---|---|---|
| Active compound | in ppm | in % after 6^{d} | |
| compound (I-2) | 0.8 | 0 | |
| compound (I-2-a) | 0.8 | 0 | |
| abamectin | 0.8 | 45 | |
| | | found* | calc.** |
| compound (I-2) + abamectin | 0.8 + 0.8 | 95 | 45 |
| (1:1) according to the invention | | | |
| | | found* | calc.** |
| compound (I-2-a) + abamectin | 0.8 + 0.8 | 90 | 45 |
| (1:1) prior art | | | |

*found = activity found
**calc. = activity calculated using Colby's formula

TABLE A2
Plant-damaging insects
Aphis gossypii test

| | Concentration | Kill | |
|---|---|---|---|
| Active compound | in ppm | in % after 6^{d} | |
| compound (I-1) | 100 | 70 | |
| compound (I-1-a) | 100 | 50 | |
| spinosad | 100 | 20 | |
| | | found* | calc.** |
| compound (I-1) + spinosad | 100 + 100 | 95 | 76 |
| (1:1) according to the invention | | | |
| | | found* | calc.** |
| compound (I-1-a) + spinosad | 100 + 100 | 70 | 60 |
| (1:1) prior art | | | |

*found = activity found
**calc. = activity calculated using Colby's formula

### EXAMPLE B

### Myzus persicae Test

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Cabbage leaves (Brassica oleracea) which are heavily infested by the green peach aphid (Myzus persicae) are treated by spraying with the active compound preparation of the desired concentration.

After the desired period of time, the kill in % is determined. 100% means that all aphids have been killed; 0% means that none of the aphids have been killed. The kill rates determined are entered into Colby's formula.

In this test, for example, the following active compound combinations in accordance with the present application show a synergistically enhanced activity compared to the active compounds applied individually:

TABLE B
Plant-damaging insects
Myzus persicae test

| | Concentration | Kill | |
|---|---|---|---|
| Active compound | in g/ha | in % after 1^{d} | |
| compound (I-2) | 20 | 0 | |
| compound (I-2-a) | 20 | 0 | |
| fenpyroximate | 20 | 20 | |
| | | found* | calc.** |
| compound (I-2) + fenpyroximate | 20 + 20 | 30 | 20 |
| (1:1) according to the invention | | | |
| | | found* | calc.** |
| compound (I-2-a) + fenpyroximate | 20 + 20 | 0 | 20 |
| (1:1) prior art | | | |
| milbemectin | 0.8 | 0 | |
| | | found* | calc.** |
| compound (I-2) + milbemectin | 20 + 0.8 | 50 | 0 |
| (25:1) according to the invention | | | |
| | | found* | calc.** |
| compound (I-2-a) + milbemectin | 20 + 0.8 | 30 | 0 |
| (25:1) prior art | | | |

*found = activity found
**calc. = activity calculated using Colby's formula

TABLE B2
Plant-damaging insects
Myzus persicae test

| | Concentration | Kill | |
|---|---|---|---|
| Active compound | in g/ha | in % after 6^{d} | |
| compound (I-2) | 0.8 | 50 | |
| compound (I-2-a) | 0.8 | 50 | |
| diafenthiuron | 4 | 60 | |
| | | found* | calc.** |
| compound (I-2) + diafenthiuron | 0.8 + 4 | 100 | 80 |
| (1:5) | | | |
| according to the invention | | | |
| compound (I-2-a) + diafenthiuron | 0.8 + 4 | 90 | 80 |
| (1:5) | | | |
| prior art | | | |

*found = activity found
**calc. = activity calculated using Colby's formula

### EXAMPLE C

### Myzus persicae Test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Cabbage leaves (Brassica oleracea) which are heavily infested by the green peach aphid (Myzus persicae) are treated by being dipped into the active compound preparation of the desired concentration.

After the desired period of time, the kill in % is determined. 100% means that all aphids have been killed; 0% means that none of the aphids have been killed. The kill rates determined are entered into Colby's formula.

In this test, for example, the following active compound combinations in accordance with the present application show a synergistically enhanced activity compared to the active compounds applied individually:

TABLE C1
Plant-damaging insects
Myzus persicae test

| | Concentration | Kill | |
|---|---|---|---|
| Active compound | in ppm | in % after 6^{d} | |
| compound (I-2) | 0.8 | 10 | |
| compound (I-2-a) | 0.8 | 0 | |
| abamectin | 0.8 | 10 | |
| | | found* | calc.** |
| compound (I-2) + abamectin | 0.8 + 0.8 | 70 | 19 |
| (1:1) | | | |
| according to the invention | | | |
| compound (I-2-a) + abamectin | 0.8 + 0.8 | 10 | 10 |
| (1:1) | | | |
| prior art | | | |
| compound (I-1) | 4 | 5 | |
| compound (I-1-a) | 4 | 20 | |
| abamectin | 4 | 65 | |
| compound (I-1) + abamectin | 4 + 4 | 98 | 66.75 |
| (1:1) | | | |
| according to the invention | | | |
| compound (I-1-a) + abamectin | 4 + 4 | 90 | 72 |
| (1:1) | | | |
| prior art | | | |

*found = activity found
**calc. = activity calculated using Colby's formula

### EXAMPLE D

### Phaedon cochleariae Larvae Test

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Cabbage leaves (Brassica oleracea) are treated by spraying with the active compound preparation of the desired concentration and are populated with larvae of the mustard beetle (Phaedon cochleariae) while the leaves are still moist.

After the desired period of time, the kill in % is determined. 100% means that all beetle larvae have been killed; 0% means that none of the beetle larvae have been killed. The kill rates determined are entered into Colby's formula.

In this test, for example, the following active compound combinations in accordance with the present application show a synergistically enhanced activity compared to the active compounds applied individually:

TABLE D1
Plant-damaging insects
Phaedon cochleariae larvae test

| | Concentration | Kill | |
|---|---|---|---|
| Active compound | in g/ha | in % after 2^{d} | |
| compound (I-2) | 20 | 0 | |
| compound (I-2-a) | 20 | 0 | |
| chlorfenapyr | 4 | 0 | |
| | | found* | calc.** |
| compound (I-2) + chlorfenapyr | 20 + 4 | 100 | 0 |
| (5:1) | | | |
| according to the invention | | | |
| compound (I-2-a) + | 20 + 4 | 67 | 0 |
| chlorfenapyr | | | |
| (5:1) | | | |
| prior art | | | |

* found = activity found
** calc. = activity calculated using Colby's formula

TABLE D2
Plant-damaging insects
Phaedon cochleariae larvae test

| | Concentration | Kill | |
|---|---|---|---|
| Active compound | in g/ha | in % after 6^{d} | |
| compound (I-2) | 20 | 0 | |
| | 4 | 0 | |
| compound (I-2-a) | 20 | 0 | |
| | 4 | 0 | |
| fenpyroximate | 20 | 33 | |
| | | found* | calc.** |
| compound (I-2) + fenpyroximate | 20 + 20 | 83 | 33 |
| (1:1) | | | |
| according to the invention | | | |
| compound (I-2-a) + fenpyroximate | 20 + 20 | 17 | 33 |
| (1:1) | | | |
| prior art | | | |
| pyridaben | 4 | 33 | |
| compound (I-2) + pyridaben | 4 + 4 | 50 | 33 |
| (1:1) | | | |
| according to the invention | | | |
| compound (I-2-a) + pyridaben | 4 + 4 | 33 | 33 |
| (1:1) | | | |
| prior art | | | |
| tebufenpyrad | 4 | 0 | |
| compound (I-2) + tebufenpyrad | 4 + 4 | 33 | 0 |
| (1:1) | | | |
| according to the invention | | | |
| compound (I-2-a) + tebufenpyrad | 4 + 4 | 0 | 0 |
| (1:1) | | | |
| prior art | | | |

*found = activity found
**calc. = activity calculated using Colby's formula

### EXAMPLE E

### Spodoptera frugiperda Larvae Test

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Cabbage leaves (Brassica oleracea) are treated by spraying with the active compound preparation of the desired concentration and are populated with larvae of the army worm (Spodoptera frugiperda) while the leaves are still moist.

After the desired period of time, the kill in % is determined. 100% means that all caterpillars have been killed; 0% means that none of the caterpillars have been killed. The kill rates determined are entered into Colby's formula (see sheet 1).

In this test, for example, the following active compound combinations in accordance with the present application show a synergistically enhanced activity compared to the active compounds applied individually:

TABLE E1
Plant-damaging insects
Spodoptera frugiperda larvae test

| | Concentration | Kill | |
|---|---|---|---|
| Active compound | in g/ha | in % after 2^{d} | |
| compound (I-2) | 100 | 83 | |
| | 20 | 17 | |
| compound (I-2-a) | 100 | 83 | |
| | 20 | 17 | |
| diafenthiuron | 100 | 50 | |
| | | found* | calc.** |
| compound (I-2) + diafenthiuron | 20 + 100 | 100 | 58.5 |
| (1:5) | | | |
| according to the invention | | | |
| compound (I-2-a) + diafenthiuron | 20 + 100 | 17 | 58.5 |
| (1:5) | | | |
| prior art | | | |
| fenpyroximate | 100 | 50 | |
| compound (I-2) + fenpyroximate | 100 + 100 | 100 | 91.5 |
| (1:1) | | | |
| according to the invention | | | |
| compound (I-2-a) + | 100 + 100 | 83 | 91.5 |
| fenpyroximate | | | |
| (1:1) | | | |
| prior art | | | |

*found = activity found
**calc. = activity calculated using Colby's formula

### EXAMPLE F

### Tetranychus Test (OP-Resistant/Spray Treatment)

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Disks of bean leaves (Phaseolus vulgaris) which are infested by all stages of the greenhouse red spider mite (Tetranychus urticae) are sprayed with an active compound preparation of the desired concentration.

After the desired period of time, the activity in % is determined. 100% means that all spider mites have been killed; 0% means that none of the spider mites have been killed.

In this test, the following active compound combination in accordance with the present application showed a synergistically enhanced activity compared to the active compounds applied individually:

TABLE F1
Plant-damaging mites
Tetranychus urticae test

| | Concentration | Kill | |
|---|---|---|---|
| Active compound | in g/ha | in % after 2^{d} | |
| compound (I-2) | 20 | 10 | |
| | 0.8 | 20 | |
| compound (I-2-a) | 20 | 0 | |
| | 0.8 | 0 | |
| pyridaben | 0.8 | 0 | |
| | | found* | calc.** |
| compound (I-2) + pyridaben | 0.8 + 0.8 | 30 | 20 |
| (1:1) | | | |
| according to the invention | | | |
| compound (I-2-a) + pyridaben | 0.8 + 0.8 | 0 | 0 |
| (1:1) | | | |
| prior art | | | |
| spinosad | 4 | 10 | |
| compound (I-2) + spinosad | 20 + 4 | 70 | 19 |
| (5:1) | | | |
| according to the invention | | | |
| compound (I-2-a) + spinosad | 20 + 4 | 20 | 10 |
| (5:1) | | | |
| prior art | | | |

*found = activity found
**calc. = activity calculated using Colby's formula

### EXAMPLE G

### Phaedon cochleariae Larvae Test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Cabbage leaves (Brassica oleracea) are treated by spraying with the active compound preparation of the desired concentration and are populated with larvae of the mustard beetle (Phaedon cochleariae) while the leaves are still moist.

After the desired period of time, the kill in % is determined. 100% means that all beetle larvae have been killed; 0% means that none of the beetle larvae have been killed. The kill rates determined are entered into Colby's formula.

In this test, for example, the following active compound combinations in accordance with the present application show a synergistically enhanced activity compared to the active compounds applied individually:

TABLE G1
Plant-damaging insects
Phaedon cochleariae larvae test

| | Concentration | Kill | |
|---|---|---|---|
| Active compound | in ppm | in % after 6^{d} | |
| compound (I-1) | 0.8 | 0 | |
| compound (I-1-a) | 0.8 | 0 | |
| spinosad | 0.8 | 20 | |
| | | found* | calc.** |
| compound (I-1) + spinosad | 0.8 + 0.8 | 50 | 20 |
| (1:1) | | | |
| according to the invention | | | |
| compound (I-1-a) + spinosad | 0.8 + 0.8 | 5 | 20 |
| (1:1) | | | |
| prior art | | | |

*found = activity found
**calc. = activity calculated using Colby's formula

### EXAMPLE H

### Critical Concentration Test/Soil Insects

### Treatment of the Transgenic Plants

| | |
|---|---|
| Test insect: | Diabrotica balteata-larvae in the soil |
| Solvent: | 7 parts by weight of acetone |
| Emulsifier: | 2 parts by weight of alkylaryl polyglycol ether |

To produce a suitable preparation of active compound, 1 part by weight of active compound is mixed with the stated amount of solvent, the stated amount of emulsifier is added and the concentrate is diluted with water to the desired concentration.

The preparation of active compound is poured onto the soil. Here, the concentration of the active compound in the preparation is virtually immaterial, only the amount by weight of active compound per volume unit of soil, which is stated in ppm (mg/l), matters. The soil is filled into 0.25 l pots, and these are allowed to stand at 20° C.

Immediately after the preparation, 5 pre-germinated maize corns of the cultivar YIELD GUARD (trademark of Monsanto Comp., USA) are placed into each pot. After 2 days, the appropriate test insects are placed into the treated soil. After a further 7 days, the efficacy of the active compound is determined by counting the maize plants that have emerged (all plants emerged=100% activity).

### EXAMPLE I

### Heliothis virescens Test

### Treatment of Transgenic Plants

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amount of solvent and the stated amount of emulsifier, and the concentrate is diluted with water to the desired concentration.

Soybean shoots (Glycine max) of the cultivar Roundup Ready (trademark of Monsanto Comp. USA) are treated by spraying with the preparation of active compound of the desired concentration and are populated with the tobacco budworm Heliothis virescens while the leaves are still moist.

After the desired period of time, the kill of the insects is determined.

### EXAMPLE J

### Myzus persicae Test

### Treatment of Transgenic Plants

| | |
|---|---|
| Solvent: | 7 parts by weight of acetone |
| Emulsifier: | 2 parts by weight of alkylaryl polyglycol ether |

To produce a suitable preparation of active compound, 1 part by weight of active compound is mixed with the stated amount of solvent and the stated amount of emulsifier, and the concentrate is diluted with water to the desired concentration.

Transgenic cabbage plants (Brassica oleracea) which are heavily infested by the green peach aphid Myzus persicae are treated by spraying with the active compound preparation of the desired concentration.

After the desired period of time, the kill of the insects is determined.

1. An active compound combination, comprising a compound of the formula (I-1) or (I-2) 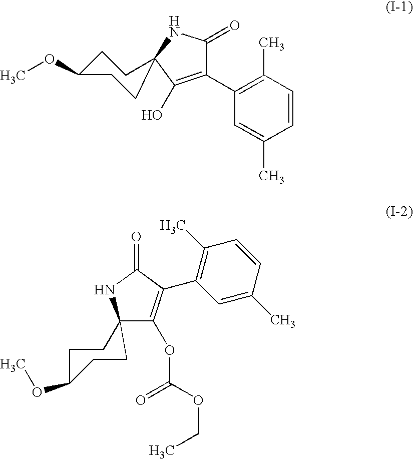
and one or more compounds selected from the group consisting of:
bifenazate,
abamectin,
acequinocyl,
chlorfenapyr,
diafenthiuron,
etoxazole,
azocyclotin,
cyhexatin,
tebufenpyrad,
fenpyroximate,
pyridaben,
clofentezine,
fenbutatin oxide,
tolylfluanid,
(4-[(4-chloro-α,α,α-trifluoro-3-tolyl)oxy]-6-[(α,α,α-4-tetrafluoro-3-tolyl)oxy]pyrimidine),
4-[(4-chloro-α,α,α-trifluoro-3-tolyl)oxy]-6-[(α,α,α-trifluoro-4-nitro-3-tolyl)oxy]pyrimidine,
4-[(4-chloro-α,α,α-trifluoro-3-tolyl)oxy]-6-[(α,α,α-trifluoro-4-bromo-3-tolyl)oxy]-pyrimidine,
spinosad,
ivermectin,
milbemectin,
fenazaquin,
pyrimidifen,
triarathen,
tetradifon,
propargite,
hexythiazox,
bromopropylate,
dicofol, and
chinomethionat.

2. The active compound combination as claimed in claim 1, comprising the compound of the formula (I-1).

3. The active compound combination as claimed in claim 1, comprising the compound of the formula (I-2).

4. (canceled)

5. A method for controlling animal pests, comprising contacting an active compound combination according to claim 1 with one or more animal pests, their habitat, or combinations thereof.

6. A process for preparing an insecticidal or an acaricidal composition, comprising mixing an active compound combination according to claim 1, with one or more extenders, surfactants, or combinations thereof.

7. A composition for controlling animal pests, comprising an active compound combination according to claim 1 and one or more extenders, surfactants, or combinations thereof.
